(19) United States
(12) Patent Application Publication
(10) Pub. No.: US 2022/0362305 A1
(43) Pub. Date: Nov. 17, 2022

(54) METHOD FOR COATING AND DRYING HETEROGENOUS STEM CELL DERIVED EXTRA-CELLULAR VESICLES

(71) Applicants: Omar Mohamed Salah Eldin Ahmed ELTOOKHY, Cairo (EG); Khaled Abou ElSouud Mahmoud IBRAHIM, Cairo (EG); Ashraf Ali Eldesouky SHAMAA, Cairo (EG); Ahmed Nour Eldine Abdallah HANAFI, Cairo (EG); Mohamed Moustafa Bahr MOUSTAFA, Cairo (EG); Mohamed Said Mostafa AMER, Cairo (EG)

(72) Inventors: Omar Mohamed Salah Eldin Ahmed ELTOOKHY, Cairo (EG); Khaled Abou ElSouud Mahmoud IBRAHIM, Cairo (EG); Ashraf Ali Eldesouky SHAMAA, Cairo (EG); Ahmed Nour Eldine Abdallah HANAFI, Cairo (EG); Mohamed Moustafa Bahr MOUSTAFA, Cairo (EG); Mohamed Said Mostafa AMER, Cairo (EG)

(21) Appl. No.: 17/755,609

(22) PCT Filed: Nov. 2, 2020
(86) PCT No.: PCT/EG2020/000030

§ 371 Date: May 3, 2022

(30) Foreign Application Priority Data
Nov. 4, 2019 (EG) 2019111752

(51) Int. Cl. A61K 35/28; C12N 5/0775; A61K 9/19

(52) U.S. Cl. CPC A61K 35/28; C12N 5/0667; C12N 5/0663; A61K 9/19; A61K 9/5015

(57) ABSTRACT

Coated and Dried extracellular vesicles (EVs) represent an ideal method for preservation and increasing the shelf life-time of the invention making it ready to use on time and on variety of species overcoming the previous challenges on isolation and preservation and undesirable immune responses. The invention compromises a coated freeze dried stem cells derived EVs that are ready for use to Stimulate/accelerate healing of soft/hard tissues, can be reconstituted in multiple forms and shapes and stimulated by Laser. The nature of the invention is a heterogenous and/or Xenogenous EVs which can overcome the challenges of individual and/or species diseases and immune reactions.

### BACKGROUND OF THE INVENTION

### 1. Field of Invention

This invention relates to a method of coating and preserving extra cellular vesicles(EVs) obtained from stem cells of heterogenous donors, facilitating the pharmaceutical manufacturing and transportation and making them ready to use.

Heterogenicity of the product refers primarily to EVs obtained from different sources/donors (human, animal, birds, eggs, primary embryonic cells), not only to different type/sizes of cells or vesicles naturally existing in extracellular solutions. Xenogenous refers to using the heterogenic product on recipient of another species/genus.

### 2. Description of Related Art

The Extracellular Vesicles (EVs) comprise all lipid bilayer-delimited particles that are naturally released from stem cells including exosomes and microvesicles which are involved in intercellular communication, immune modulation, senescence, proliferation and differentiation among various processes (Nassar, 2014 Extracellular Vesicles (EVs); Basic Science, Clinical Relevance and Applications).

EVs derived from stem cells hold a great regenerative capacity because it contains the major beneficial cytokines and growth factors secreted by stem cells (Katsuda and Ochiya, 2015 Molecular signatures of mesenchymal stem cell-derived extracellular vesicle-mediated tissue repair).

Studies have shown that EVs have the same regenerative role as the cells of origin on damaged tissues.

EVs help to overcome problems of cell therapy, such as: tumor formation and the rejection due to undesirable immune reaction of the recipient's body.(Sabin, K; Kikyo, N 2014:Microvesicles as mediators of tissue regeneration).

It has not been tested among individuals of the same species or between different species and genera. (Fuster et al., 2015: Acellular approaches for regenerative medicine: on the verge of clinical trials with extracellular membrane vesicles?).

Moreover, experimental studies of the invention showed antiapoptotic, antifibrotic and angiogenic activities beside the positive stimulation on the endogenous stem/progenitor cells creating a regenerative milieu.

### 3. Technical Problems

There is no instant, ready-to-use product.

Currently, EVs are prepared from one individual and are used for the same individual.

It takes 3-6 weeks to prepare a new preparation from the same individual and requires the availability of special laboratory tools.

The fresh product cannot be kept at normal room temperature and cannot be kept viable for longer than 7 days.

Previous preparations existed in liquid form only.

Previous preparations use was limited to adminstration by injection.

### SOLUTION TO PROBLEMS

Invention is ready for immediate use.

Nature (Heterogenous/Xenogenous): extracting and preserving high grade EVs from an individual and using them in another individual of the same or different species/genus.

Saves time of preperation

Long shelf life, preservation in room temperature and nature.

The preparation can be manufacutred in various forms.

The preparation can be administered via various routes.

### 4. Advantageous Effects of Invention

The ease of collecting and preparing the product saving time and effort.

Can be safely carried on many natural and synthetic vehicles.

The possibility of controlling the degree of hardness/viscosity.

The invention has an identified protein content and can be produced in different concentrations and all possible pharmaceutical forms.

The product can be kept at normal room temperature and for much longer times than the freshly prepared one.

Administration varies according to pharmaceutical formand to the target site of treatment.

Immediate intervention avoiding delays in culture and processing which are crucial in progressive diseases therapy.

EVs perform the same work as the cells of origin and as efficiently as the freshly prepared one despite being dehydrated.

Stimulate/accelerate healing of soft/hard tissues for example, treat skin problems/wounds, heart problems, musculoskeletal degenerative disorders, hepatic and renal degenerative disorders, immune stimulation/modulation and tissue grafts.

EVs perform the same work as the cells of origin and as efficiently as the freshly prepared one despite being dehydrated.

Can be used for studies in vivo and in vitro for evaluation of biological experiments like cell signaling, proteomics, cellular proliferation etc

Does not generate an immune reaction.

### DETAILED DESCRIPTION OF INVENTION

The invention compromises a method of preparation and preservation of Stem cell derived EVs to obtain a ready to use high grade concentrates of growth factors and cytokines to promote the healing of soft/hard tissues and can be used in different pharmaceutical shapes/forms with a long shelf life. And detailed as follows:

The product is prepared from a donor (human/animal) and is used in another recipient (human/animal) without any immune reaction. It can be used among different species and genera and is not limited as the previous preparations.

The EVs are loaded onto coating materials of different cyto-compatible natures.

The vesicles-loaded coating material is exposed to a drying process to extract water particles from it and convert it into solid/powder.

Preservation in room temperature without damage/reduced efficiency of the vesicles.

The final product can be prepared/shaped according to the required method of use (e.g. not limited to: fluids for injection by different routes or for oral intake in the form of tablets, pills, syrup, or the like). As well as local use as drops, ointments, or gels, or included in other compounds such as external dressings.

With the possibility of using it without an immune reaction, this guarantees the production of commercial quantities for use among humans and animals.

In addition, the coating and drying of the vesicles help to: (a) Protect the vesicles during the drying process, (b) Give the vesicles a degree of viscosity that enables them to hold together and bond with the tissue when treating wounds or diluting it according to the purpose of use, (c) When reconstituting the dried material, it returns to its original form immediately without significant change in the degree of viscosity, (d) Coating ensures that the outer cellular vesicles are preserved without a change in their therapeutic efficiency, (e) The ease of shaping the invention into different pharmaceutical forms (liquid/powder/tablets . . . ) that is appropriate to the place to be treated, (f) Drying makes it easy to produce, transport and use therapeutically and commercially.

### Laboratory Experiments

Animal study (I) of reconstituted EVs on superficial wounds proved to: (a) Increase the speed of wound healing. (b) The healed tissue is of a better quality (less fibrous tissue), no scar. (c) Compared to commercial skin healing products it had a superior healing power regarding the healing time and quality. (d) in vitro analysis of the composition showed no significant change in physical or biological properties of the dried EVs.

Animal study (II) to evaluate the effect of Stem cell derived EVs in repair of induced chondral defects in a dog model showed that administration of EVs was effective on the functional and morphological recovery of the injured cartilage and could be exploited as a cell free therapeutic approach in regenerative medicine to achieve the restoration of chondral histomorphological picture within a period of 3 months with mature collagen fibers on histopathological evaluation on the contrary of the control joints that showed deterioration over time and defect filling with only fibrous tissue forming a fibrocartilage.

Side effects: No known side effects of the invention.

Method of exploitation (a) The dried product loaded with the EVs is reconstituted by distilled water or similar solvent. (b) The product returns to its original form immediately without any significant change and the required liquidity/viscosity. (c) The invention can be prepared and packaged in the form of tablets/capsules or similar to be taken orally or for the place to be treated (ointment/injection/oral/ . . . ).

### Effectiveness

EVs perform the same work as the cells of origin and as efficiently as the freshly prepared one despite being dehydrated.

Stimulate/accelerate healing of soft/hard tissues for example, treat skin problems/wounds, heart problems, musculoskeletal degenerative disorders, hepatic and renal degenerative disorders, immune stimulation/modulation and tissue grafts.

Can be used for studies in vivo and in vitro for evaluation of biological experiments like cell signaling, proteomics, cellular proliferation etc.

Does not generate an immune reaction.

### Expected Outcomes

Compared to the stem cell transplants, the invention holds the similar quality of healing.

Compared to conventional drugs, the invention can be used to achieve quicker and better healing quality of soft tissues/bones.

Absence of side effects such as, cell-toxicity, infection, immune reactions, or immune rejection.

What is claimed:

1. A Lyophilized composition comprising stem cells (SCs) derived extra-cellular vesicles (EVs) obtained from heterogeneous donors.

2. A Lyophilized composition according to claim 1, wherein vesicles are coated by a protective substance to preserve their effectiveness.

3. A Lyophilized composition according to claim 1, wherein vesicles are further dried to improve storage time/conditions and facilitate the end-product formulation and transportation.

4. A Lyophilized composition according to claim 1, wherein vesicles may be used as is, mixed or added to other ingredient(s) to reach a specific biological and/or therapeutic effect(s), not limited to herbs, antibiotics, vitamins, or other pharmaceutical or biological ingredients. etc. to enhance/increase/add extra therapeutic/biological properties.

5. A Lyophilized composition according to claim 1, wherein vesicles combined with other polymers to produce biologically enhanced polymers.

6. A Lyophilized composition according to claim 1, wherein vesicles from heterogeneous donors are cross-used among different species (Xenogenously).

7. Method of preparation of Lyophilized composition according to claim 1, wherein the method comprises the following: (a) Bone marrow/adipose tissue is obtained from human, different animal species (dogs, cats, horses, ruminants, etc.). (b) SCs are isolated from bone marrow/adipose tissue in lab. (c) Isolated SCs are cultured for 3 to 4 weeks in incubators. (d) SCs are left to secrete EVs in a preconditioned media for 1 week. (e) Collection of EVs from the conditioned media using ultracentrifugation or fractionation.

8. Method of preparation of Lyophilized composition according to claim 7, wherein the method further comprise coating of extracellular vesicles (EVs) obtained/collected from a single and/or multiple sources “donor”, of a single and/or multiple species; by a protective substance to preserve their effectiveness and storage time using any of the following substances (e.g. and not limited to):
(a) Polysaccharides, such as and not limited to starch, cellulose, gum, glycogen, gelatin, pectin, dextrin, alginate, chitosan. (b) Glycoprotein such as selectin, vegetable or mineral oils and fats such as lanolin and similar animal products such as egg white, milk or their natural or industrial derivatives. (c) Acids derived from sugars such as glycolic acid, tartaric, citric acid. (d) Liposomes (e) Nanoparticles and Nano carriers. (f) Natural or industrial plastics and polyether compounds such as Polyethylene glycol. (g) Therapeutic compositions: salt solutions/phosphate buffer saline, anti-inflammatories such as DMSO and the like.

9. Method of preparation of Lyophilized composition according to claim 7, wherein the method further comprise drying of the extracellular vesicles (EVs) obtained/collected from a single and/or multiple sources “donor”, of a single and/or multiple species; by means of (and not limited to) Lyophilization, Spray drying, Microwave assisted drying, Annealing, Desiccation and Vacuum dehydration.

10. Method of preparation according to claim 7 wherein the method comprises biological stimulation of the end-product (Kit) by laser.
(a) The kit can be stimulated by low level laser exposure to increase the potency and proliferation. (b) The kit is exposed to wave low level laser with power density of 5.5 mW/cm² and a wavelength of 635 nm for 20 min

11. Kit comprising composition according to claim 1, wherein the kit comprises Lyophilized composition can be formulated in different shapes for use according to the reconstitution method:
(a) Reconstituted with little amount of sterile water or normal saline to give the shape of a viscous gel or ointment. (b) Further dilution to obtain a watery solution (drops or injection). (c) Watery solution packed in atomizer (spray or inhalation). (d) The diluted form can be electro-spun with other polymers to give different shapes and sizes like rods, threads, tablets, hydrogels, etc (for implantation). (e) The dried product is added to adhesive bandages as a wound/burn skin dressing.

12. Kit comprising composition according to claim 1, wherein the kit comprises Lyophilized composition collected from different donors (human or animals) is used among different recipients of the same species as a “Heterogenous” population. The “Heterogenous” population of the collected EVs is used also among different species “Xenogenous” e.g. From dogs to cats.
